(19) United States
(12) Patent Application Publication
(10) Pub. No.: US 2021/0353960 A1
(43) Pub. Date: Nov. 18, 2021

(54) DEVICE FOR SHIELDING IMPLANTABLE RADIOACTIVE SOURCES TO ACHIEVE DIRECTIONAL DOSING

(71) Applicant: Isoray Medical, Inc., Richland, WA (US)

(72) Inventors: Troy SIENKO, Richland, WA (US); William A. CAVANAGH, III, Richland, WA (US)

(73) Assignee: Isoray Medical, Inc., Richland, WA (US)

(21) Appl. No.: 17/318,356

(22) Filed: May 12, 2021

Related U.S. Application Data
(60) Provisional application No. 63/024,062, filed on May 13, 2020.

(51) Int. Cl. A61N 5/10

(52) U.S. Cl. CPC A61N 5/1007; A61N 2005/1094; A61N 2005/101

(57) ABSTRACT

A cradle made of radiation-shielding material holds one or more radioactive seeds. The cradle surrounds all but a portion of the seed, and the potion of the cradle that does not surround the seeds creates an aperture through which the radiation escapes the cradle. The cradle reflects and absorbs radiation from the seed, resulting in directional dosing toward diseased tissue and away from healthy tissue. In a preferred embodiment the cradle has four walls and a bottom, forming a cavity into which the seed is secured with biocompatible epoxy. The side opposite the bottom is typically open, but may instead be made of a material that is transparent to radiation. The cradle wall thickness, bottom thickness, and cavity height determine the direction, shape, and intensity of the radiation dispersion. Cradles may be attached to a biocompatible mesh to form a sheet with directional radiation.

### CROSS-REFERENCE TO RELATED APPLICATIONS

This application claims the benefit of co-pending U.S. Provisional Application No. 63/024,062 filed May 13, 2021.

### FIELD OF INVENTION

This invention relates generally to devices for brachytherapy. More particularly, this invention relates to devices for shielding implantable radioactive sources to achieve directional dosing, particularly at the interface between healthy and diseased tissue.

### BACKGROUND

Brachytherapy is the treatment of cancer by the insertion of radioactive implants directly into the tissue near the tumor. The implants are minute radioactive pellets known as seeds. Seeds are radioactive sources covered in a biocompatible shell. The seeds may be implanted individually or inserted into a suture material which is sewn in place in tissue. Seeds, and, optionally, non-radioactive pellets known as spacers, may be lined up end-to-end in strands that are held together in a sleeve and secured by plugging the ends of the sleeve with bone wax. The loaded sleeve is then placed in a needle and inserted into the patient's tissue at the desired location. Alternatively, the seeds may be embedded into a mesh or sponge-like material, and the mesh implanted into the patient's tissue. Seeds are chosen such that they lose their radioactivity after the dose is complete so seeds that remain in the body are inert. Depending on the type of cancer and patient conditions, brachytherapy can be performed with radioactive seeds that remain in the body permanently or only temporarily.

Brachytherapy seeds come in many different isotopes, including gold-198, iridium-192, iodine-125, palladium-103 and cesium-131. Different isotopes have different radiation effects, including the intensity of the radiation, the distance it penetrates and the length of time the isotope is actively emitting radiation. The isotope is chosen for its radiation energy, intensity and half-life properties. Low dose-rate brachytherapy is a common treatment where the seeds put out a small amount of radiation over a duration of several weeks to months. High dose-rate brachytherapy procedures last only a few minutes, and commonly the radioactive material is removed at the end of the treatment session. For example, cesium-131 can deliver a high dose over a period of 30 to 45 days, maximizing dose and coverage while minimizing treatment length.

Brachytherapy is used to treat many types of cancers, including brain, head and neck, lung, breast, prostate, and gynecological cancers including cervical, ovarian, uterine, vaginal, and vulvar. Radioactive isotopes contained within the seed emit radiation in an isotropic manner, in all directions, even though the radiation-transparent shell that encases the isotopes. That means that radiation sources placed near tumors irradiate not only the tumor, but healthy tissue nearby including muscles, ligaments, and organs. For example, to treat prostate cancer with a prostate seed implant, typically all areas of the prostate are covered with seeds. That means that sensitive areas outside the prostate such as bladder, rectum, urethra and nerves run the risk of being irradiated too. The position of many tumors relative to sensitive surrounding organs requires precise delivery of the radiation to limit toxicity to the nearby organs. It would be desirable to shield healthy tissue from radiation.

It would be advantageous to have an implantable brachytherapy source that emits directional radiation to treat diseased tissue while sparing healthy tissue from radiation.

### SUMMARY OF THE INVENTION

The present invention is a cradle made of radiation-shielding material which holds one or more radioactive seeds. The cradle surrounds all but a portion of the seed, and the portion of the cradle that does not surround the seeds creates an aperture through which the radiation escapes the cradle. The cradle reflects and absorbs radiation from the seed, resulting in directional dosing toward diseased tissue and away from healthy tissue. In a preferred embodiment the cradle has four walls and a bottom, forming a cavity into which the seed is secured with biocompatible epoxy. The side opposite the bottom is typically open, but may instead be made of a material that is transparent, or semi-transparent, to radiation. The cradle wall thickness, bottom thickness, and cavity height determine the direction, shape, and intensity of the radiation dispersion. Cradles may be attached to a biocompatible mesh to form a sheet with directional radiation.

### BRIEF DESCRIPTION OF THE DRAWINGS

FIG. 1 is a top view of a radioactive seed of the prior art.

FIG. 2 is an end cross section view of the seed taken along line 2-2 of FIG. 1.

FIG. 3 is a top cross section view of the seed taken along line 3-3 of FIG. 1.

FIG. 4 is an end cross section view of the cradle taken along line 4-4 of FIG. 5.

FIG. 5 is a top view of a cradle of the present invention.

FIG. 6 is an end view of the seed and cradle of FIG. 7.

FIG. 7 is a top view of the radioactive seed in the cradle of the present invention.

FIG. 8 is an end cross section view of the seed in the cradle taken along line 8-8 of FIG. 7.

FIG. 9 is a top cross section view of the seed in the cradle taken along line 9-9 of FIG. 7.

FIG. 10 is a graph showing the radiation dispersal of a 2.5 U Cs-131 seed without a cradle, where the seed is centered at the origin y=0, z=0 and the seed extends in the x-axis into the page.

FIG. 11 is a graph showing the radiation dispersal of a seed of FIG. 10 in a cradle with a cavity height to the top of the seed, wall thickness of 0.60 mm, and bottom thickness of 0.60 mm.

FIG. 12 is a graph showing the radiation dispersal the seed of FIG. 10 in Co—Cr cradle having cavity height 0.84 mm, wall thickness 0.20 mm and bottom thickness of 0.20.

FIG. 13 is a graph showing the radiation dispersal the seed of FIG. 10 in Co—Cr cradle having cavity height 0.84 mm, wall thickness 0.30 mm, and bottom thickness of 0.30 mm.

FIG. 14 is a graph showing the radiation dispersal the seed of FIG. 10 in Co—Cr cradle having cavity height 0.84 mm, wall thickness 0.40 mm, and bottom thickness of 0.40 mm.

FIG. 15 is a graph showing the radiation dispersal the seed of FIG. 10 in Co—Cr cradle having cavity height 0.84 mm, wall thickness 0.50 mm, and bottom thickness of 0.50 mm.

FIG. 16 is a graph showing the radiation dispersal the seed of FIG. 10 in Co—Cr cradle having cavity height 0.84 mm, wall thickness 0.60 mm, and bottom thickness of 0.60 mm.

FIG. 17 is a graph showing the radiation dispersal the seed of FIG. 10 in Co—Cr cradle having cavity height 0.68 mm, wall thickness 0.50 mm, and bottom thickness of 0.50 mm.

FIG. 18 is a graph showing the radiation dispersal the seed of FIG. 10 in Co—Cr cradle having cavity height 0.44 mm, wall thickness 0.50 mm, and bottom thickness of 0.50 mm.

FIG. 19 is a graph showing the radiation dispersal the seed of FIG. 10 in Co—Cr cradle having cavity height 0.20 mm, wall thickness 0.50 mm, and bottom thickness of 0.50 mm.

FIG. 20 is a graph showing the radiation dispersal the seed of FIG. 10 in Co—Cr cradle having cavity height 0.0 mm and bottom thickness 0.50 mm.

FIG. 21 is a top perspective view of twenty-five seed-loaded cradles attached to a biocompatible mesh, using the seed of FIG. 10 in a Co—Cr cradle having cavity height 0.44 mm, wall thickness 0.50 mm and bottom thickness of 0.50 mm.

FIG. 22 shows the radiation dispersal of the seed-loaded cradle assemblies of FIG. 21 on a y-z plane at x=0 and the origin at the center of the middle seed.

FIG. 23 is an end view of FIG. 21.

FIG. 24 is a graph showing a top view of the radiation dispersal of the seed-loaded cradle assemblies of FIG. 21 with the long side of the seeds along the x-axis, viewed with the z-axis going into the page on the x-y plane at z=5.0 mm.

FIG. 25 is a graph showing an end view of the radiation dispersal of the seed-loaded cradle assemblies of FIG. 21 on a y-z plane at x=0 and the origin at the center of the middle seed.

FIG. 26 is a graph showing the radiation dispersal of the treatment for rectal cancer described in Example 1, using a 5×10 array of 50 cradle assemblies.

FIG. 27 is a graph showing the radiation dispersal of the seeds of FIG. 26 if no cradles are used.

FIG. 28 is a graph showing the radiation dispersal of the treatment for rectal cancer described in Example 2, using a 6×12 array of 72 cradle assemblies.

FIG. 29 is a graph showing the radiation dispersal of the seeds of FIG. 28 if no cradles are used.

FIG. 30 is a graph showing the radiation dispersal of the treatment for rectal cancer described in Example 2, using an 8×8 array of 64 cradle assemblies.

FIG. 31 is a graph showing the radiation dispersal of the seeds of FIG. 30 if no cradles are used.

### DETAILED DESCRIPTION OF THE INVENTION

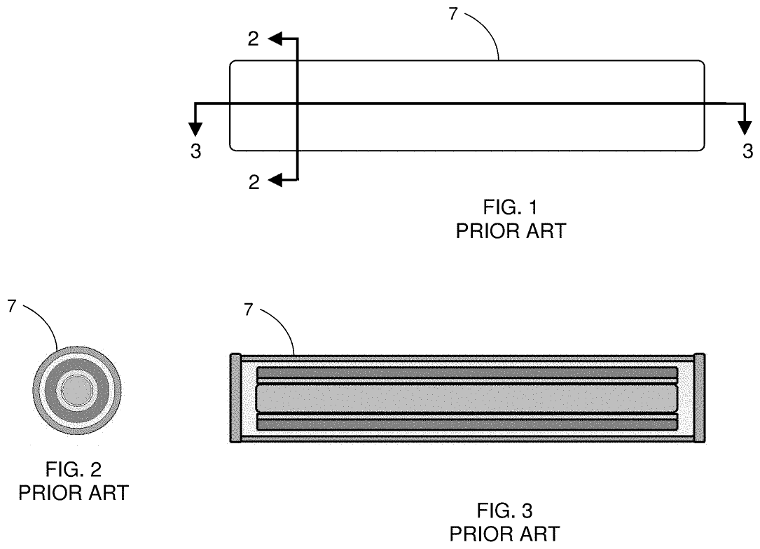

FIGS. 1-3 illustrate a typical brachytherapy seed 7 of the prior art. The seeds are radioactive and may be made of any radioactive isotope. The most common radioisotopes used for permanent implant brachytherapy include I-125, Cs-131, Pd-103 and Y-90.

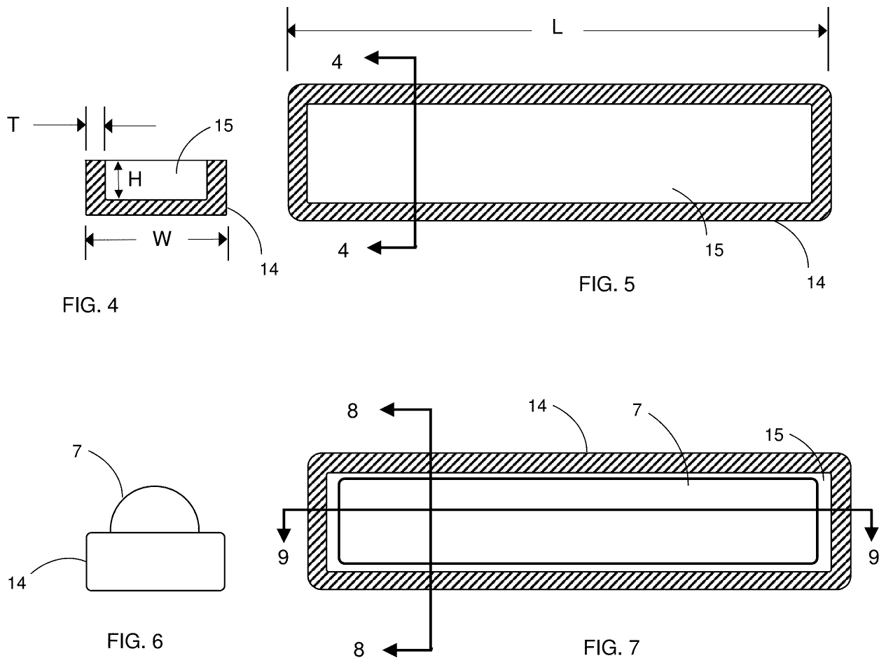

As used herein, a cradle 14 is a solid mass that has a cavity 15 therein sized to accommodate one or more seeds. The cradle may have different external shapes depending on the need, such as a sphere, a hemisphere or half-round, a quarter round, an ovoid, a square box, or other shape. In a preferred embodiment, the external shape of a cradle 14 is like an open rectangular box, having four walls and a bottom, forming an internal rectangular cavity 15 into which the seed 7 is placed and secured. The side opposite the bottom is typically completely open, forming a 5-sided box. See FIGS. 4-5. Alternatively, the side opposite the bottom may partially enclose the cavity or may instead be made of a material that is transparent or semi-transparent to the energy of the treatment radiation. The cradle 14 may be manufactured with any cavity 15 size to accommodate any size brachytherapy seed containing any isotope.

The cradle provides shielding, as opposed to the shielding being a part of the radiation source itself. That is, the radiation shield is the cradle in which the seed is disposed, as opposed to the shield being integral with the seed. The cradle may hold high-dose rate or low-dose rate radiation sources. The cradle does not pass through a needle. Instead, the loaded cradles are implanted into the patient. The cradles may themselves be attached to the patient's tissue or the cradles may be attached to a substrate that is attached to the patient's tissue.

Changing cradle wall or cradle bottom thickness or cradle cavity height changes the direction, shape and intensity of the radiation dispersion. For rectangular cradles, cradle wall thickness, cradle bottom thickness, and cradle cavity height determine the direction and shape of the radiation dispersion. Cradle wall thickness T is typically the same for all walls and the bottom, but certain walls or the bottom, or both, may have different thicknesses in certain applications. Cradle cavity height H is typically the same for all cavity walls, but certain walls may have different heights in certain applications.

The width of the cavity 15 inside the cradle 14 is the inside-inside width between the walls of the cradle 14, which for walls of the same thickness can be calculated as the width of the cradle 14 W minus (2×cradle 14 wall thickness T). The length of the cavity 15 is the inside-inside length of the cavity which, for walls of the same thickness, can be calculated as the length of the cradle 14 L minus (2×cradle 14 wall thickness T). The depth of the cavity 15 can be calculated as the height of the cradle 14 H minus the bottom thickness.

Table 1 is an example of the dimensions of a cradle 14 and cavity 15 formed therein, where the cradle 14 wall and bottom thickness T is 0.200 mm.

TABLE 1

| | Cradle | | | |
|---|---|---|---|---|
| | 14 | | | Wall and |
| | Width, | Cradle 14 | Cradle 14 | Bottom |
| | W | Length, L | Height, H | Thickness |
| Cradle 14 Dimensions | 1.400 | 5.000 | 0.620 | 0.200 |
| (mm) | | | | |
| Cavity 15 Dimensions (mm) | 1.000 | 4.600 | 0.420 | NA |

Table 2 shows the dimensions of commercially available Cs-131 seeds, available commercially from Isoray, Inc.

TABLE 2
Cs-131 overall dimensional tolerances

| L min | L max | OD min | OD max |
|---|---|---|---|
| 3.999 | 5.019 | 0.813 | 0.863 |

The cavity 15 is sized to receive the seed, and the tolerance between the seed and cavity is sized to permit easy manufacture. In one embodiment, the cavity 15 is sized to provide 0.1 mm space between the seed 7 and each wall, when the seed 7 is centered in the cavity. The cavity width and length are generally fixed, but the cradle outside dimensions vary around the fixed cavity size, given the changes to cradle wall thickness, bottom thickness and wall height desired to change the shape and direction of the emitted dose.

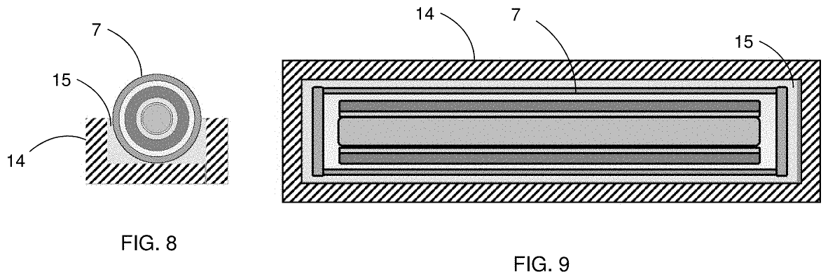

The cradle may take on other form factors to accommodate other seed shapes and doses, especially as such form factors are approved for use by the FDA. The cradles may be manufactured in bulk to accommodate standard seed sizes, for example by additive manufacturing, molding, casting or milling. FIGS. 6-9 illustrate the seed 7 in the cradle 14, each referred to herein as a cradle assembly 17. In a preferred embodiment, a single cradle is sized and shaped to accommodate all FDA-approved seeds.

The cradle wall and bottom are made from biocompatible material that blocks at least the energy or energies of the treatment radiation emitted from the seed. These materials are referred to herein as radiopaque materials. For example, x-rays are blocked by radiation-shielding material such as metals including Co—Cr, Co—Cr alloys, and Au Iridium, Platinum, Tantalum, Tungsten or other suitable biocompatible material. Radiopaque materials may prevent or permit transmission of wavelengths that are different from the wavelength or wavelengths of the treatment radiation emitted from the seed. The cradle may be made of a radiopaque material that is biodegradable or radiation-degradable material and, once degraded, leaves the seeds behind. In such case the rate of degradation is chosen such that it occurs after the seed's emissions are so low as to not damage nearby tissue that was originally shielded. Materials that are conducive to constructing cradles by additive manufacturing are advantageous, including Co—Cr, Co—Cr alloys, Au—Ir, Pt, Ta, and W. Non-metal biocompatible materials may be used to make the cradle.

The walls and bottom of the cradle should be substantially inflexible, which means that while there may be a limited amount of flex in the cradle material, the cradle should stand upright with the seed in it. In a preferred embodiment, the cradle walls and bottom are rigid.

The cradle retains the seed in the cavity such that the seed is not accidentally dislocated from the cradle during shipping, implantation, or after implantation. The seed is retained in the cradle either permanently or for a period of time sufficient for the seed's radiation levels to be low enough to not damage healthy tissue. In one embodiment, biocompatible medical-grade epoxy is used to permanently attach the seed 7 to the cradle 14 in the cavity 15. Other biocompatible adhesives may be used. Alternatively or in combination with the adhesive, a cradle may be sized to permit a friction fit or snap fit between the seed and the cradle to retain the seed in the cradle.

In some embodiments a radiographic marker, such as a gold wire, dot or other structure that is visible during medical imaging may be incorporated into the cradle to increase visibility of the devices during medical imaging.

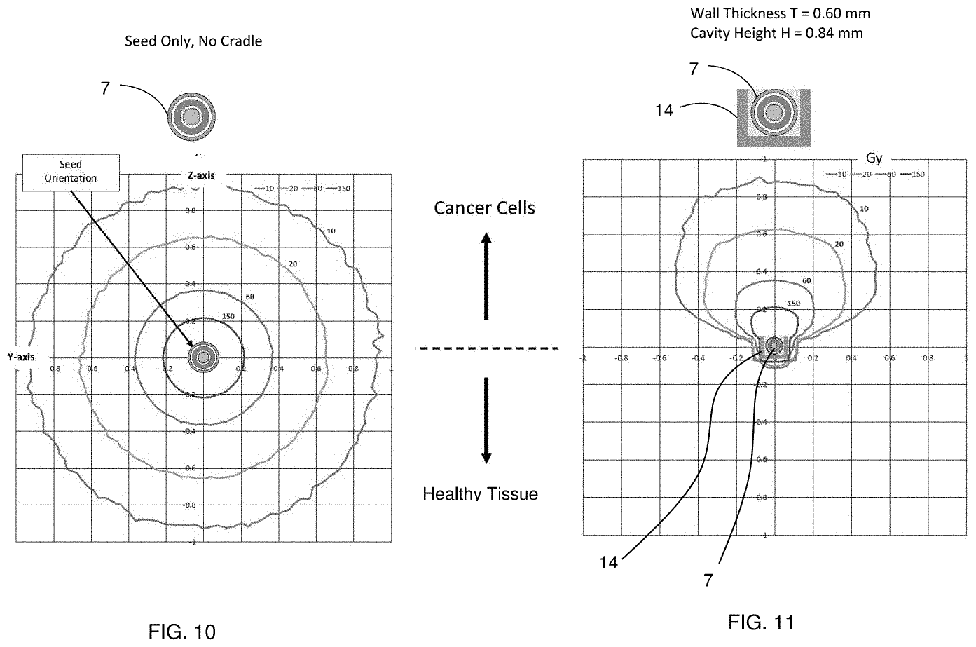

FIGS. 10-20 show the total dose absorbed in Gy at the indicated distance from the origin. Each grid line denotes 0.2 cm distance from the origin. In each of these FIGS. 10-20, the seed is centered at the origin where y=0, z=0.

FIG. 10 shows a standard seed isodose profile of a 2.5 U Cs-131 seed with no cradle 14, centered at the origin where y=0, z=0, with the seed extending in the x-axis into the page. The dose is distributed uniformly in all directions, with equal dosing at equal distances from the origin. FIG. 11 shows the isodose profile resulting from the same 2.5 U Cs-131 seed in a Co—Cr cradle 14 where the cavity height reaches the top of the seed. For example, for a standard seed of 0.84 mm diameter, and wall thickness of 0.5 mm the cavity height is 0.84 mm.

The resulting isodose lines show that for the seed in the cradle the dose is distributed asymmetrically away from the origin, with the dose distributed relatively evenly in one direction while blocked in the other. By placing the seed-loaded cradle assembly 17 with the cavity opening toward the tumor and the cradle bottom toward the healthy tissue, the cancer tissue is irradiated while the healthy tissue is spared.

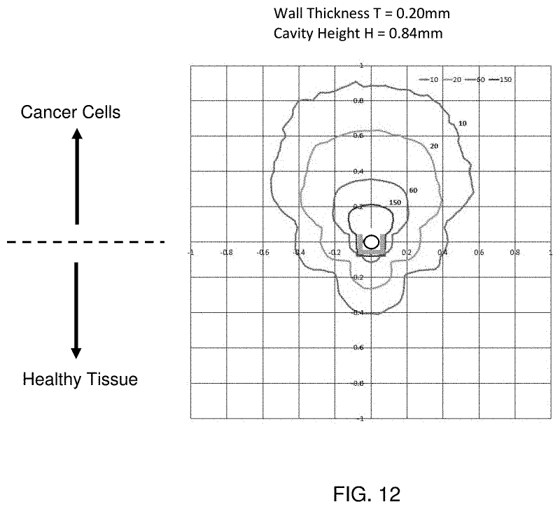

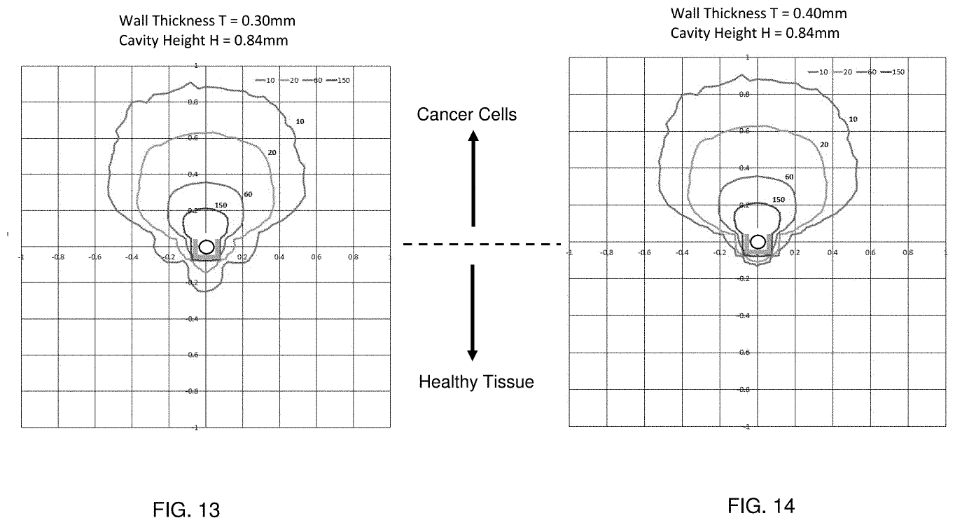

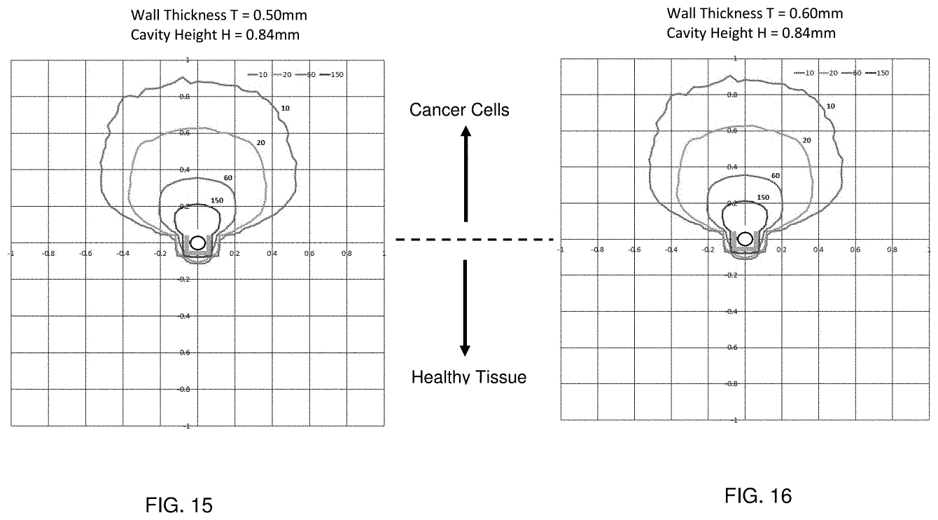

FIGS. 12-16 show a cradle 14 wall thickness T variation simulation to optimize shielding and delivered dose for the 2.5 U Cs-131 seed of FIG. 10 in a cradle 14 with a constant cavity height of 0.84 mm. The wall thickness T progresses from 0.20 mm in FIG. 12 to 0.30 mm in FIG. 13, to 0.40 mm in FIG. 14, to 0.50 mm in FIG. 15, and to 0.60 mm in FIG. 16. The bottom thickness is the same as the wall thickness T in each of FIG. 12-16.

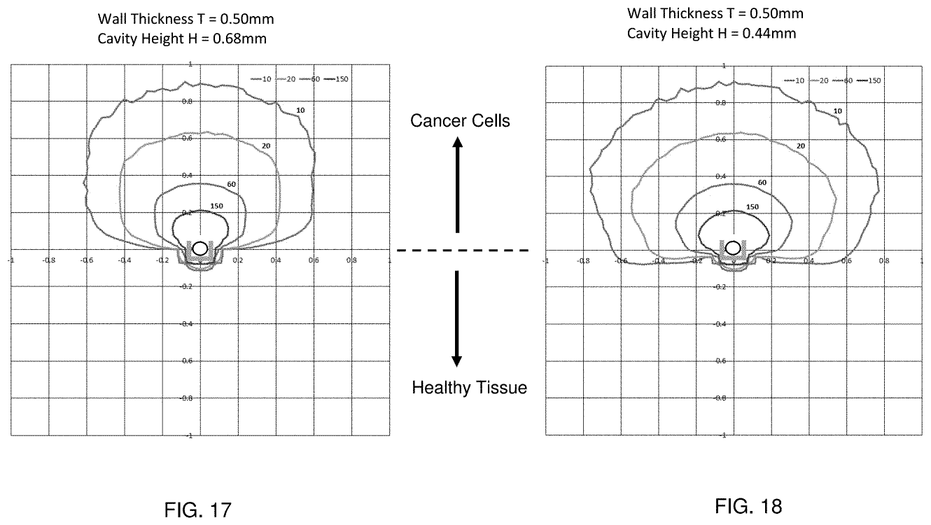

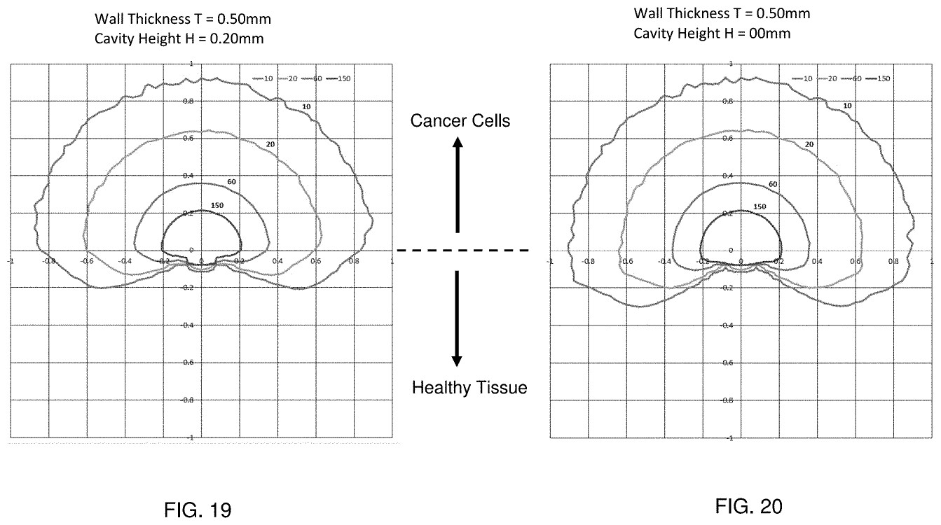

FIGS. 15, 17-20 show a cradle 14 cavity height H variation simulation to optimize shielding and delivered dose for the 2.5 U Cs-131 seed of FIG. 10 in a cradle 14 with a constant wall thickness of 0.5 mm. The cavity height H progresses from 0.84 mm in FIGS. 15 to 0.68 mm in FIG. 17, 0.44 mm in FIG. 18, 0.20 mm in FIG. 19, 0.00 mm in FIG. 20. The bottom thickness is the same as the wall thickness T in each of FIG. 15, 17-20.

To prevent migration of individual seeds and cradle combinations, the seed-loaded cradle assemblies 17 may be attached to a substrate 19, such as a biocompatible mesh or a wafer that is similar to a surgical sponge. In a preferred embodiment, the substrate 19 is bendable but resilient, so that a piece of the substrate with cradle assemblies 17 attached can be gently folded or rolled up to a smaller size for implantation and then unfurled to its original shape once placed in the body at the desired location. The biocompatible mesh may be cut to customize a shape of the mesh for implantation.

In some embodiments that mesh is radio-transparent and provides no shielding in addition to the cradle. In other embodiments, the mesh itself may provide additional shielding, under the cradles or adjacent to them or both.

The mesh is preferably a biocompatible non-resorpable metal that does not degrade when subject to radiation, such as titanium mesh which is available commercially in standard sizes. Alternatively, the mesh may be biodegradable and, once degraded, leaves the cradle-seed assemblies behind.

In one example, the substrate is a 6″×6″ bioabsorbable knitted flat mesh of Vicryl® (Polyglactin 910) prepared from the synthetic bioabsorbable copolymer 10% polylactide (PLA) and 90% polyglycolide (PGA). The mesh is compatible with ethylene oxide sterilization, non-reactive, biocompatible and is bioabsorbable within approximately 56-70 days post-implant. This mesh is available commercially.

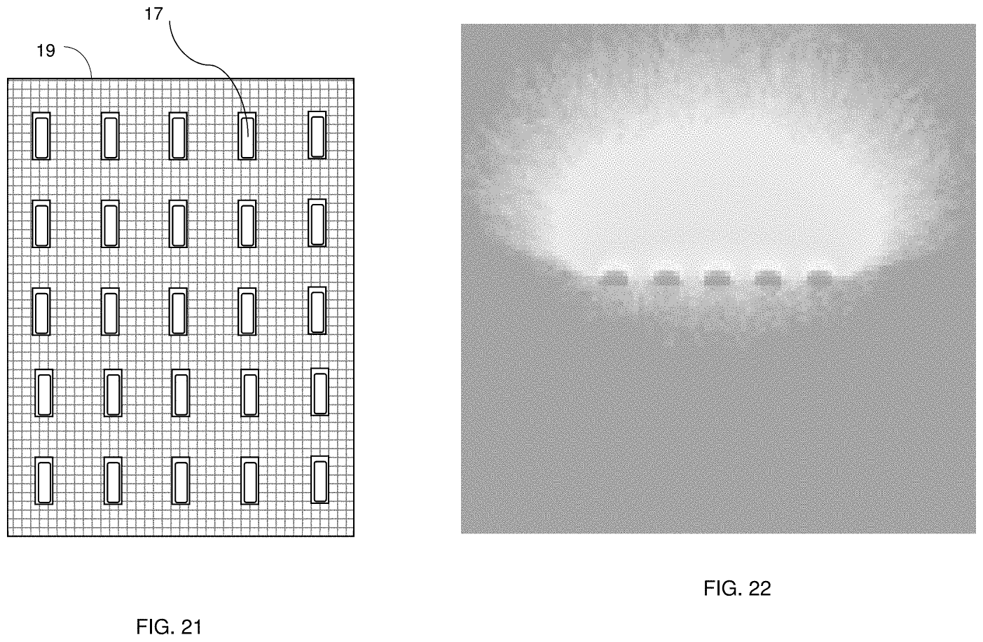

The cradle assemblies 17 are preferably attached to the mesh by weld, sutured or biocompatible epoxy. In a preferred embodiment, the bottom of the cradle 14 is attached to the mesh so that there is shielding between the seed and the mesh and the radiation is emitted generally away from the mesh. FIG. 21 shows twenty-five seed-loaded cradle assemblies attached to a mesh. The biocompatible mesh holds multiple radiation sources in precise positions. The radiation sources may be of similar type or different type, depending on the location and dosage required. The cradle assemblies may be uniformly or non-uniformly spaced, depending on the location and dosage required.

Alternatively, a wall of the cradle 14 is attached to the mesh so that there is shielding between the seed and the mesh and the radiation is emitted generally perpendicularly away from the mesh. In another version, the open side of the cradle 14 is attached to the mesh so that there is no shielding between the seed and the mesh and the radiation is emitted generally perpendicularly towards the mesh. Mesh may be attached to either the cancerous tissue or healthy tissue.]

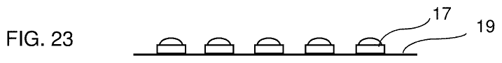

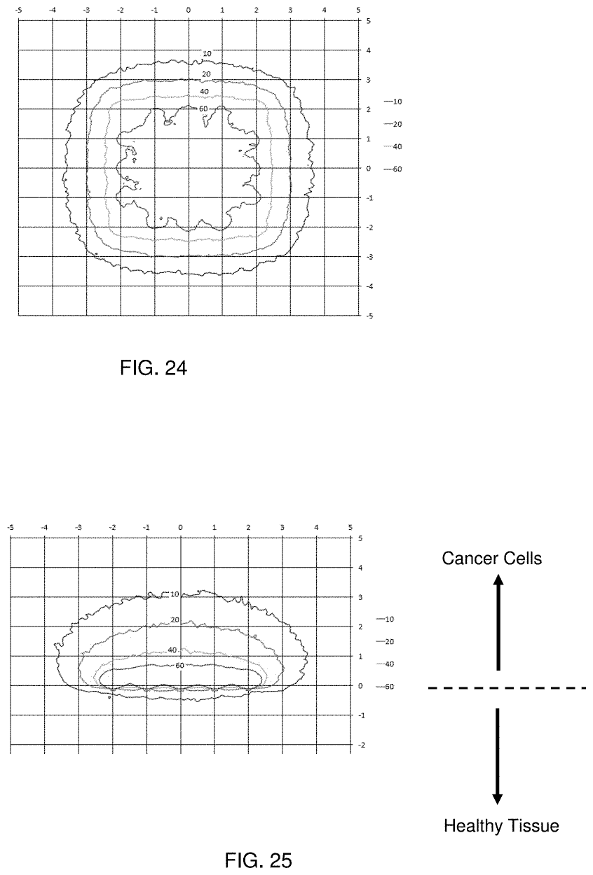

FIG. 24 shows the radiation dispersal of twenty-five 2.5 U Cs-131 seeds with cradles arranged in a 5-by-5 array with the long side along the x-axis, viewed with the z-axis going into the page on the x-y plane at z=5.0 mm. FIG. 23 shows five seed-loaded cradle assemblies 17 on a mesh 19. FIGS. 22 and 25 shows the radiation dispersal of twenty-five seed-loaded cradle assemblies of FIG. 21. Again, the resulting isodose lines show that for the seeds in cradles the dose is distributed asymmetrically away from the origin, with the dose distributed relatively evenly in one direction while blocked in the other. By placing the mesh with the cavity openings toward the resected tumor and the cradle bottom toward the healthy tissue, the cancerous tissue is irradiated while the healthy tissue is not.

Instead of using a mesh to prevent migration of individual seeds, the seed-loaded cradle assemblies 17 may instead be sutured or adhered to a patient's tissue. Individual cradles or chains of cradles may be sutured or adhered in place.

### Treatment Example 1

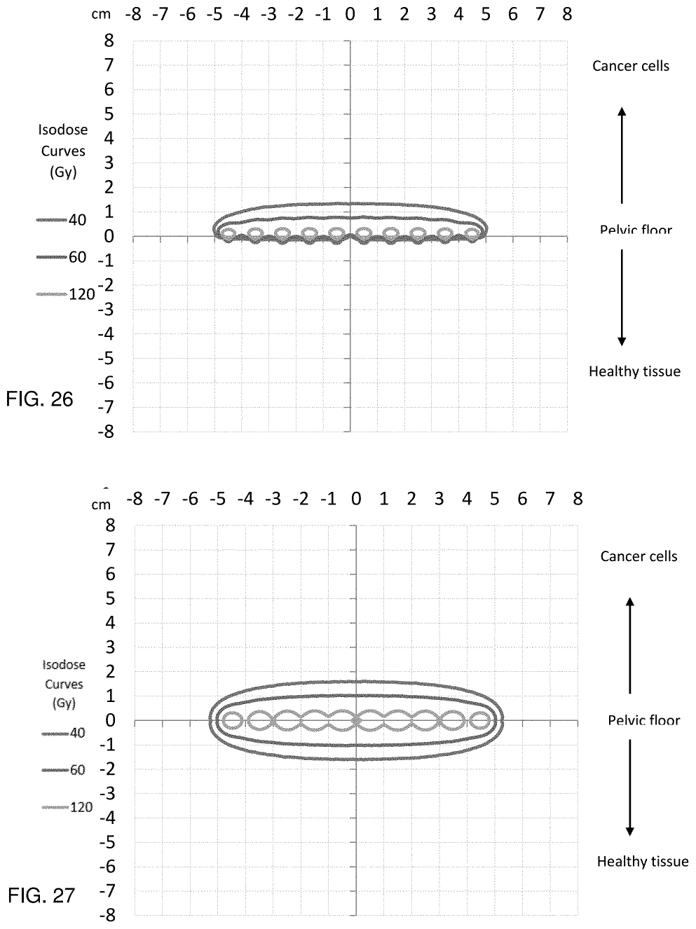

A patient is diagnosed with rectal cancer invasive to the pelvic floor. The patient's physician develops a radiation treatment plan having a prescribed dose of 60 Gy at a tissue depth of 5 mm. This is accomplished using 50 cradle assemblies. Each cradle is made of Co—Cr, with a 1.0 mm wall and bottom thickness and a cavity height of 0.48 mm. Each cradle loaded with a Cs-131 seed with an air kerma strength of 2.4 U. The cradle assemblies are attached in a 5×10 array to a 6 cm×11 cm rectangle of mesh, evenly distributed with 1 cm on-center spacing on the x-y plane. Note that the treatment is dependent on the array of cradles and independent of the size of the mesh to which they are attached, which may be trimmed to a desired size for attachment to the patient's tissue. The mesh is sutured to the patient's pelvic floor, with the therapeutic radiation directed toward the pelvic floor (down), sparing the intestines from radiation exposure. FIG. 26 illustrates a MCNP6 Monte Carlo simulation of the dose attenuation and tissue-sparing effect using the loaded cradle assemblies set forth in this Example 1, as compared to FIG. 27 which illustrates the dose if cradles are not used.

### Treatment Example 2

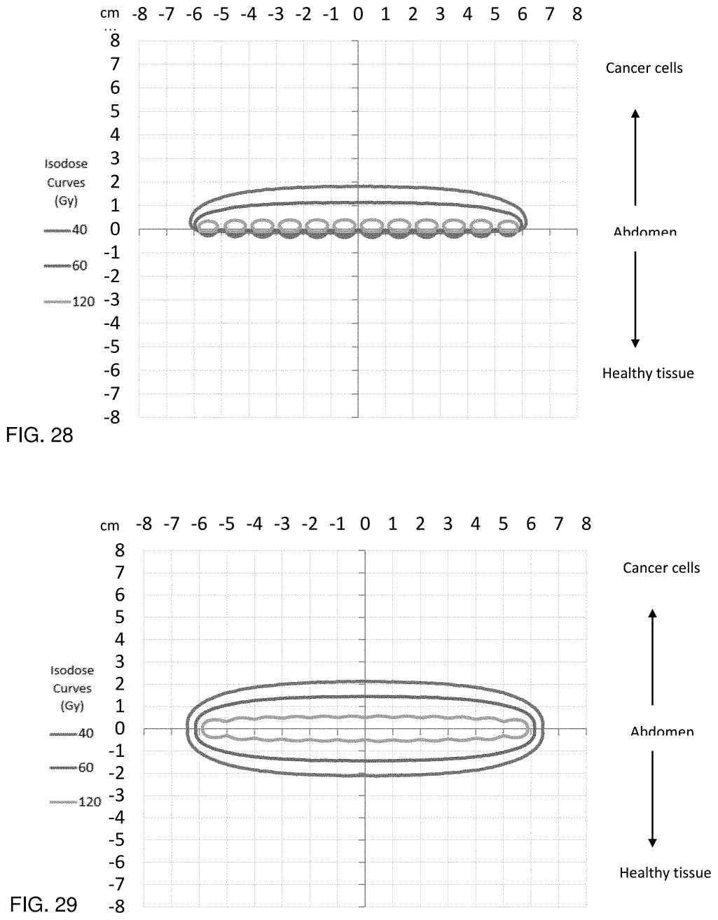

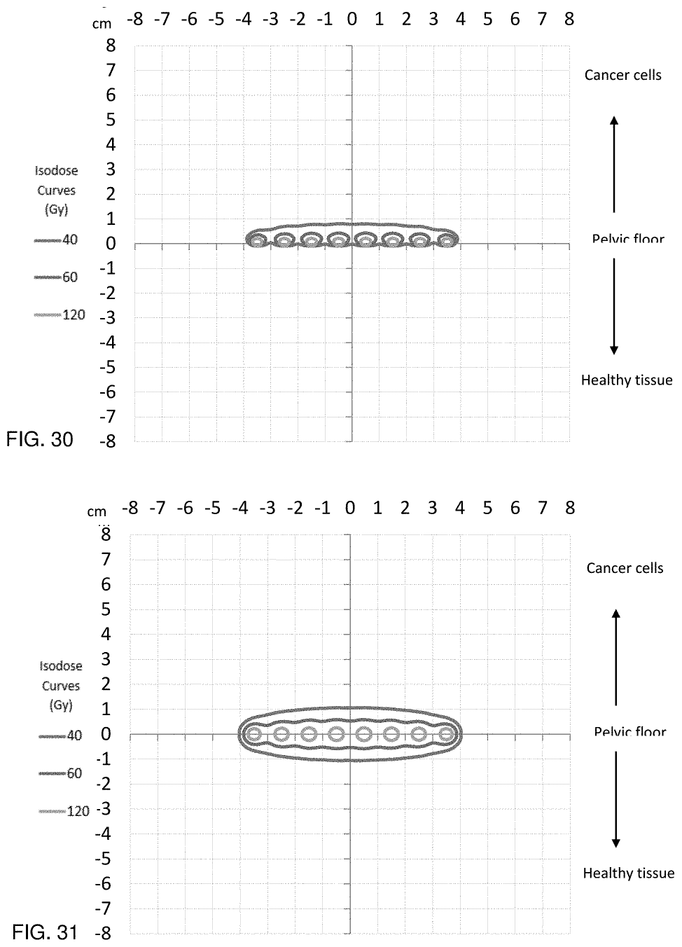

A patient is diagnosed with retroperitoneal carcinoma. The patient's physician develops a radiation treatment plan having a prescribed dose of 60 Gy at a tissue depth of 10 mm. This is accomplished using 72 cradle assemblies. Each cradle is made of Co—Cr, with a 0.20 mm wall and bottom thickness and a cavity height of 0.52 mm. Each cradle loaded with a Cs-131 seed with an air kerma strength of 3.0 U. The cradle assemblies are attached in a 6×12 array to a 7 cm×13 cm rectangle of mesh, evenly distributed with 1 cm on-center spacing on the x-y plane. Note that the treatment is dependent on the array of cradles and independent of the size of the mesh to which they are attached, which may be trimmed to a desired size for attachment to the patient's tissue. The mesh is sutured to the patient's abdomen, with the therapeutic radiation directed toward the abdominal wall, sparing the intestines from radiation exposure. FIG. 28 illustrates a simulation of the dose attenuation and tissue-sparing effect using the loaded cradle assemblies set forth in this Example 2, as compared to FIG. 29 which illustrates the dose if cradles are not used.

### Treatment Example 3

A patient is diagnosed with ovarian cancer with pelvic floor invasion. The patient's physician develops a radiation treatment plan having a prescribed dose of 40 Gy at a tissue depth of 5 mm. This is accomplished using 64 cradle assemblies. Each cradle is made of Tantalum, with a 0.10 mm wall thickness, 0.15 mm bottom thickness, and a cavity height of 0.42 mm. Each cradle loaded with a Cs-131 seed with an air kerma strength of 1.5 U. The cradle assemblies are attached in an 8×8 array to a 9 cm×9 cm rectangle of mesh, evenly distributed with 1 cm on-center spacing on the x-y plane. Note that the treatment is dependent on the array of cradles and independent of the size of the mesh to which they are attached, which may be trimmed to a desired size for attachment to the patient's tissue. The mesh is sutured to the patient's pelvic floor, with the therapeutic radiation directed toward the pelvic floor (down), sparing the intestines from radiation exposure.

While there has been illustrated and described what is at present considered to be the preferred embodiment of the present invention, it will be understood by those skilled in the art that various changes and modifications may be made and equivalents may be substituted for elements thereof without departing from the true scope of the invention. Therefore, it is intended that this invention not be limited to the particular embodiment disclosed, but that the invention will include all embodiments and equivalents falling within the scope of the appended claims.

We claim:

1. A directional dosing device for brachytherapy, the device comprising:
a. a cradle comprising a cavity sized to accommodate a radioactive seed, wherein the cradle is made of a radiopaque material; and
b. an aperture in the cradle to receive the seed into the cavity and to enable directional radiation emission from the seed.

2. The directional dosing device of claim 1 further comprising a mesh substrate to which one or more cradles are attached.

3. The directional dosing device of claim 1 wherein the cradle has four walls and a bottom forming a rectangular cradle with a rectangular cavity.

4. The directional dosing device of claim 1 wherein the radiopaque material is a metal or a metal alloy.

5. The directional dosing device of claim 1 wherein the radiopaque material is Co—Cr or Co—Cr alloy.

6. The directional dosing device of claim 1 wherein the radiopaque material is not metal.

7. The directional dosing device of claim 1 wherein the radioactive seed comprises Cs-131, Au-198, Ir-192, I-125, or Pd-103.

8. A directional dosing device for brachytherapy, the device comprising:
a. a solid mass of radiopaque material having a cavity sized to accommodate a radioactive seed; and
b. an aperture from the cavity to outside the mass to enable directional radiation emission from the seed.

9. The directional dosing device of claim 8 wherein the solid mass has an aperture to receive the seed.

10. The directional dosing device of claim 8 wherein the radiopaque material is conducive to additive manufacturing.

11. The directional dosing device of claim 8 wherein the radiopaque material is a metal or a metal alloy.

12. The directional dosing device of claim 8 wherein the radiopaque material is Co—Cr or Co—Cr alloy.

13. The directional dosing device of claim 8 wherein the radiopaque material is not metal.

14. The directional dosing device of claim 8 wherein the radioactive seed comprises Cs-131.

15. A directional dosing assembly for brachytherapy comprising:
a. a radioactive seed; and
b. a cradle comprising a cavity holding the radioactive seed, wherein:
i. the cradle is made of a radiopaque material; and
ii. the cradle further comprises an aperture through which radiation is emitted from the seed.

16. The directional dosing assembly of claim 15 wherein the seed is held in the cradle with a biocompatible epoxy.

17. The directional dosing assembly of claim 15 further comprising a mesh substrate to which one or more cradles are attached.

18. The directional dosing device of claim 15 wherein the radiopaque material is a metal or a metal alloy.

19. The directional dosing device of claim 1 wherein the radiopaque material is Co—Cr or Co—Cr alloy.

20. The directional dosing device of claim 8 wherein the radioactive seed comprises Cs-131.
